(19) United States
(12) Patent Application Publication
(10) Pub. No.: US 2023/0240641 A1
(43) Pub. Date: Aug. 3, 2023

(54) SYSTEM AND METHOD FOR DETERMINING AN AUSCULTATION QUALITY METRIC

(71) Applicant: THE JOHNS HOPKINS UNIVERSITY, Baltimore, MD (US)

(72) Inventors: Mounya ELHILALI, North Potomac, MD (US); Annapurna KALA, Baltimore, MD (US)

(73) Assignee: THE JOHNS HOPKINS UNIVERSITY, Baltimore, MD (US)

(21) Appl. No.: 18/004,966

(22) PCT Filed: Jun. 28, 2021
(86) PCT No.: PCT/US2021/039309

§ 371 Date: Jan. 10, 2023

Related U.S. Application Data
(60) Provisional application No. 63/053,472, filed on Jul. 17, 2020.

(51) Int. Cl. A61B 7/00; A61B 5/00

(52) U.S. Cl. CPC A61B 7/003; A61B 5/7221

(57) ABSTRACT

A computer-implemented method, a computer system, and a non-transitory computer readable medium are provided that perform a method for determining an auscultation quality metric (AQM). The computer-implemented method includes obtaining an acoustic signal representative of pulmonary sounds from a patient; determining a plurality of derived signals from the acoustic signal; performing a regression analysis on the plurality of derived signals; and determining the AQM from the regression analysis.

### CROSS-REFERENCE TO RELATED APPLICATIONS

This application claims priority to U.S. provisional patent application No. 63/053,472 filed on Jul. 17, 2020, which is hereby incorporated by reference in its entirety.

### GOVERNMENT RIGHTS

This invention was made with government support under Grant Nos. HL133043 and AG058532 awarded by the National Institutes of Health. The government has certain rights in the invention.

### FIELD

The present teachings generally relate to characterizing sound quality for lung auscultations.

### BACKGROUND

A stethoscope is considered the most basic tool to listen to sounds from the chest for the detection of lung and heart conditions, including diseases, since the 1800s. However, it remains a limited tool despite numerous attempts at reinventing the technology, due to major shortcomings including the need for a highly trained physician or medical worker to properly position it and interpret the auscultation signal as well as masking effects by ambient noise particularly in unusual clinical settings such as rural and community clinics. With advances in health technologies including digital devices and new wearable sensors, access to these sounds is becoming easier and abundant; yet proper measures of signal quality do not exist. Moreover, with advances in telemedicine, digital health, and use of digital stethoscopes, lung auscultations (i.e. recordings of lung sounds) provide recordings of the sound and are becoming part of the digital health record. Yet, there are no methods that currently exist to perform quality control on these recordings. Listening to a body sound can be often masked by ambient noises which are often picked up by the stethoscope or recording device.

### SUMMARY

In accordance with examples of the present disclosure, a computer-implemented method for determining an auscultation quality metric (AQM) is disclosed. The computer-implemented method comprises obtaining an acoustic signal representative of pulmonary sounds from a patient; determining a plurality of derived signals from the acoustic signal; performing a regression analysis on the plurality of derived signals; and determining the AQM from the regression analysis.

Various additional features of the computer-implemented method can include one or more of the following features. The plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, a low modulation rate energy signal, or various combinations thereof. The mean error signal and the reconstruction error signal are obtained from a trained neural network. In some examples, the trained neural network can be a trained convolutional autoencoder. The trained neural network can comprise three layers or other configurations autoencoders. The computer-implemented can further comprise training a convolutional autoencoder from a set of high-quality acoustic signals obtained from a variety of patients. The AQM ranges from 0 to 1 where 0 represents the lowest quality and 1 represents the highest quality for the acoustic signal that is obtained.

In accordance with examples of the present disclosure, a computer system is disclosed. The computer system comprises a hardware processor; a non-transitory computer readable medium comprising instructions that when executed by the hardware processor perform a method for determining an auscultation quality metric (AQM), comprising: obtaining an acoustic signal representative of pulmonary sounds from a patient; determining a plurality of derived signals from the acoustic signal; performing a regression analysis on the plurality of derived signals; and determining the AQM from the regression analysis.

Various additional features of the computer system can include one or more of the following features. The plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, a low modulation rate energy signal, or various combinations thereof. The mean error signal and the reconstruction error signal are obtained from a trained neural network. In some examples, the trained neural network can be a trained convolutional autoencoder. The trained neural network can comprise three layers or other configurations autoencoders. The hardware processor is further configured to execute the method comprising training a convolutional autoencoder from a set of acoustic signals obtained from a variety of patients. The AQM ranges from 0 to 1 where 0 represents the lowest quality and 1 represents the highest quality for the acoustic signal that is obtained.

In accordance with examples of the present disclosure, a non-transitory computer readable medium is disclosed that comprises instructions that when executed by a hardware processor perform a method for determining an auscultation quality metric (AQM), method comprising: obtaining an acoustic signal representative of pulmonary sounds from a patient; determining a plurality of derived signals from the acoustic signal; performing a regression analysis on the plurality of derived signals; and determining the AQM from the regression analysis.

Various additional features of the non-transitory computer readable medium can include one or more of the following features. The plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, a low modulation rate energy signal, or various combinations thereof. The mean error signal and the reconstruction error signal are obtained from a trained neural network. In some examples, the trained neural network can be a trained convolutional autoencoder. The trained neural network can comprise three layers or other configurations autoencoders. The method further comprises training a convolutional autoencoder from a set of acoustic signals obtained from a variety of patients. The AQM ranges from 0 to 1 where 0 represents the lowest quality and 1 represents the highest quality for the acoustic signal that is obtained.

### BRIEF DESCRIPTION OF THE FIGURES

FIG. 1 shows a plot of frequency vs time for abnormal lung spectrogram.

FIG. 2 shows a plot of frequency vs time for noisy normal lung spectrogram.

FIG. 3 shows a method of data preparation, according to examples of the present disclosure.

FIG. 4 show the processing using a convolutional autoencoder to produce the mean error μ and the reconstruction error ω, according to examples of the present disclosure.

FIG. 5A and FIG. 5B show embedded features across different SNR values, according to examples of the present disclosure.

FIG. 6 shows a regression block diagram, according to examples of the present disclosure.

FIG. 7 shows a block diagram 700 of the signal-derived regression parameters of FIG. 6.

FIG. 8 shows the linear regression on log transform and the auscultation quality metric of FIG. 6 in more detail.

FIG. 9 shows a plot of linear regression weight versus features.

FIG. 10 shows average Auscultation Quality Metric (AQM) from 0 to 1 vs signal to noise ratio (SNR) in dB with the circles indicating the SNR values included in the Γ_{Regression}^{Train}. The error bars represent variance of AQM for each SNR, according to examples of the present disclosure.

FIG. 11 show a method for determining an auscultation quality metric (AQM), according to examples of the present disclosure.

FIG. 12 is an example of a hardware configuration for a computer device, which can be used to perform one or more of the processes described above.

### DETAILED DESCRIPTION

Generally speaking, examples of the present disclosure provide for an objective quality metric of lung sounds based on low-level and high-level features in order to independently assess the integrity of the signal in presence of interference from ambient sounds and other distortions. The disclosed quality metric outlines a mapping of auscultation signals onto rich low-level features extracted directly from the signal which capture spectral and temporal characteristics of the signal. Complementing these signal derived attributes, high-level learnt embedding features are disclosed that are extracted from an auto-encoder trained to map auscultation signals onto a representative space that best captures the inherent statistics of lung sounds. Integrating both low-level (signal-derived) and high-level (embedding) features yields a robust correlation of 0.89 to infer expected quality level of the signal at various signal-to-noise. The disclosed method is validated on a large dataset of lung auscultation recorded in various clinical settings with controlled varying degrees of noise interference.

Recording and storing the lung sounds digitally paved the way to the development of computer-aided analyses in the field of auscultation. Several studies were focused on detecting adventitious breathing patterns. Proper profiling of these pathological indicators could eventually be used in diagnosing pulmonary diseases thereby potentially substituting trained personnel in the lack of medical expertise.

New deep learning approaches have opened a lot of possibilities in fields like computer vision and speech recognition exploiting the availability of large amounts of data. Access to data can also promote use of artificial-intelligence tools to aid diagnostics, telemedicine and computer-aided healthcare. In the domain of digital auscultations, the issue of data access and curation remains a limiting factor. While there are numerous studies that analyze lung sounds in laboratory settings or controlled environments, study conditions limit their applicability to real-life clinical conditions. Specifically, lung sounds collected in busy clinical settings tend to vary highly depending on the surrounding conditions at the time of recording. Additionally, the differences in devices and sensors themselves exacerbate variability in the data collected. Ultimately, there are no agreed-upon standards as to what constitutes “good data” in the domain of digital auscultations.

This disclose provides for an objective metric of the quality of a lung sound. It is noted that the metric is not an indicator of the presence or absence of adventitious lung sounds lending to the diagnosis or classification of lung sounds. Instead, the objective metric aims to deliver an independent assessment of the integrity of the lung signal and whether it is still valuable as an auscultation signal or whether it has been masked by ambient sounds and distortions which would render it uninterpretable to the ears of a physician or to an automated classification system.

One of the challenges for developing such metrics is the properties of breathing patterns like wheezes and crackles. In addition to covering a large frequency span of 50 to 2500 Hz between the two, these abnormal lung sounds often masquerade as noise. Any objective metric obtained should be careful about not misinterpreting such cases as low-quality. In this disclosure, such a metric is provided by working with both normal and abnormal lung sounds regarded to be of high quality by medical experts.

The disclosed system and method provide for a determination of a metric to assess the quality of a recording of lung sounds (obtained using a stethoscope). The metric offers an independent assessment of the integrity of the lung signal and whether it is still valuable as an auscultation signal; or whether it has been masked by ambient sounds and distortions which would render it uninterpretable to the ears of a physician or to an automated classification system.

The disclosed system and method process recordings of lung sounds and objectively assesses their quality. The disclosed system and method can be combined with digital stethoscopes where patients are asked to upload a recording of breathing from their lungs for their physicians to assess remotely and automated apps to perform computer-aided pulmonology diagnosis. The software can be used as a triage tool to flag out low-quality recordings.

Recording and storing lung sounds digitally for computer-aided analyses need high quality data. Conditions in busy clinical settings degrade quality of lung sounds (background chatter, electronic buzz, sometimes even heartbeat). Differences in source environment (ambulance, ER/OR, rural clinic) exacerbate variability in data collected. Necessity for agreed-upon standards as to what constitutes “good auscultation data.”

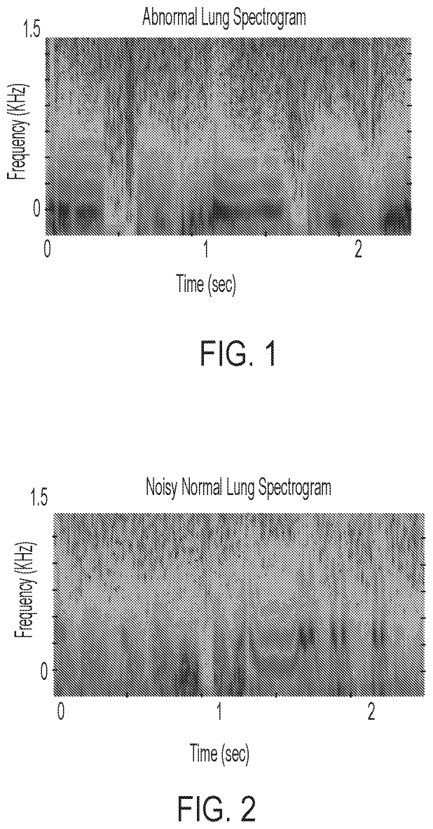

There are challenges in characterizing auscultation quality metric. For example, abnormal lung sounds frequency ranges from 50 to 2500 Hz which makes defining a quality metric not so simple. Noise can often sound similar to abnormal lung sounds. Mistaking abnormality (which is a pathological indicator) as noise should be minimized. FIG. 1 shows a plot of frequency vs time for abnormal lung spectrogram. FIG. 2 shows a plot of frequency vs time for noisy normal lung spectrogram.

In order to characterize and determine the auscultation quality metric, data is gathered and pre-processed prior to the modelling. For example, a digital stethoscope can be used for collecting lung sounds from one or more body positions. Clinical settings where data is being collected can pose a number of challenges. Lung sounds can be masked by ambient noises such as background chatter in the waiting room, vehicle sirens, mobile or other electronic interference. The data can be collected at a variety of sampling frequencies, such as at 44.1 KHz. As part of preprocessing, the data can be filtered using a low-pass filter with a fourth-order Butterworth filter at 4 kHz cutoff, down sampled to 8 kHz, and centered to zero mean and unit variance. The data can be further enhanced to deal with clipping distortions, mechanical or sensor artifacts, heart sound's interference, and ambient noise.

For an experimental study conducted by the inventors, 250 hours of recorded lung sounds were extracted from a dataset that were annotated by a panel of 9 expert listeners (pediatricians or pediatric-experienced physicians). Only segments for which a majority of expert listeners agreed on the clinical diagnosis (as normal or abnormal) with high confidence were kept. This curated subset of the data was considered to be a ‘High Quality’ database of auscultation signals for which there was a clear medical agreement from expert physicians on the patient's condition. This is referred to as a high-quality dataset collected in an everyday clinical settings as Γ_{HQ}. It included data from around 900 pediatric patients and contained an equal number of normal cases (no acute lower respiratory infections) and abnormal cases (signals containing crackles and wheezing which reflect acute lower respiratory infections including pneumonia).

To systematically vary the quality of this clean dataset, these auscultations signals were corrupted with ambient noises at controlled signal-to-noise (SNR) levels. Background noises consisted of sounds obtained from the BBC sound effects database, and included 2 hours of chatter and crowd sounds which comprised of wide range of noises like children crying, background conversations, footsteps and electronic buzzing. These BBC sounds effects signals were chosen as they offer non-stationary ambient sounds that reflect changes that can be encountered in everyday environments including clinical settings.

The entire Γ_{HQ} dataset was divided into Γ_{HQ}^{Train} and Γ_{HQ}^{Test} in a 80-20 ratio such that both datasets have equal number of normal and abnormal lung sounds. Γ_{HQ}^{Train} dataset was used to learn the profile of high quality lung sounds in an unsupervised fashion. Γ_{HQ}^{Test} was added to the BBC ambient sounds with varying signal-to-noise ratios ranging between −10 dB and 36 dB to obtain Γ_{Noisy} on which the quality metric was estimated.

A regression model is provided which estimates a quality metric based on the extent of corruption. For this purpose, a dataset Γ_{Regression}^{Train} is formed comprising 80% of Γ_{Noisy} having signal to noise ratios −5 dB, 10 dB and 20 dB. And to get a sense of perfect score, 80% of Γ_{HQ}^{Test} is included in it. The performance of the regression model is tested on Γ_{Regression}^{Test} included the other 20% of Γ_{HQ}^{Test} as well as 20% of Γ_{Noisy} across all the signal to noise ratios ranging from −10 to 36 dB.

An objective quality metric for lung sounds is provided which accounts for masking from ambient noise but is robust to the presence of adventitious lung sounds which are pathological indicators of the signal rather than a sign of low quality. A wide set of low-level and high-level features are considered in order to profile a clean lung sound (including both normal and abnormal cases), as outlined next.

In order to estimate a quality metric, the following features were extracted from auscultation signals in Γ_{Noisy} dataset. The first set of features includes spectrotemporal features. An acoustic analysis of each auscultation signal was performed as follows: The time signal is first mapped to a time-frequency spectrogram using an array of spectral filters. This spectrogram is then used to extract nine spectral and temporal characteristics of the signal, which include the following. Average spectral energy (E[S(f)]): This feature is obtained by averaging the expectation of energy content in the adjacent frequency bins of an auditory spectrogram. Pitch (\hat{F}₀): This fundamental frequency was calculated by matching the spectral profile of each time slice to a best fit from a set of pitch templates and estimating a maximum likelihood method to fit a pitch frequency to selected template. Rate Average Energy (E[\hat{R}(f)]): This feature represents the average of temporal energy variations along each frequency channel over a range of 2 to 32 Hz. Scale Average Energy (E[Ŝ(f)]): These modulations capture the average of energy spread in the spectrogram over a bank of log-spaced spectral filters ranging between 0.25 and 8 cycles/octave. Bandwidth (BW): This feature is computed as the weighted distance of the spectral profile from its centroid. Spectral Flatness (SF): This property of the spectrum is captured as the geometric mean of the spectrum divided by its arithmetic mean. Spectral Irregularity (SI): These modulations of signal are calculated as the difference in strength between adjacent frequency channels. High Modulation Rate Energy (HR): This feature captures the roughness of the signal and is obtained by the energy content in the modulation frequencies above 30 Hz. Low Modulation Rate Energy (LR): This feature is obtained as the energy content in modulation frequencies from 1 to 30 Hz.

The second set of features includes unsupervised embedding features. A convolutional neural network autoencoder can be trained in an unsupervised fashion on Γ_{HQTrain} dataset to obtain profile of high-quality lung sounds which were considered clinically highly interpretable. As this dataset has equal number of normal and abnormal lung sounds, adventitious breathing patterns get represented as part of the ‘high-quality’ lung sound templates learned by the network; and are not considered as indicators of poor quality.

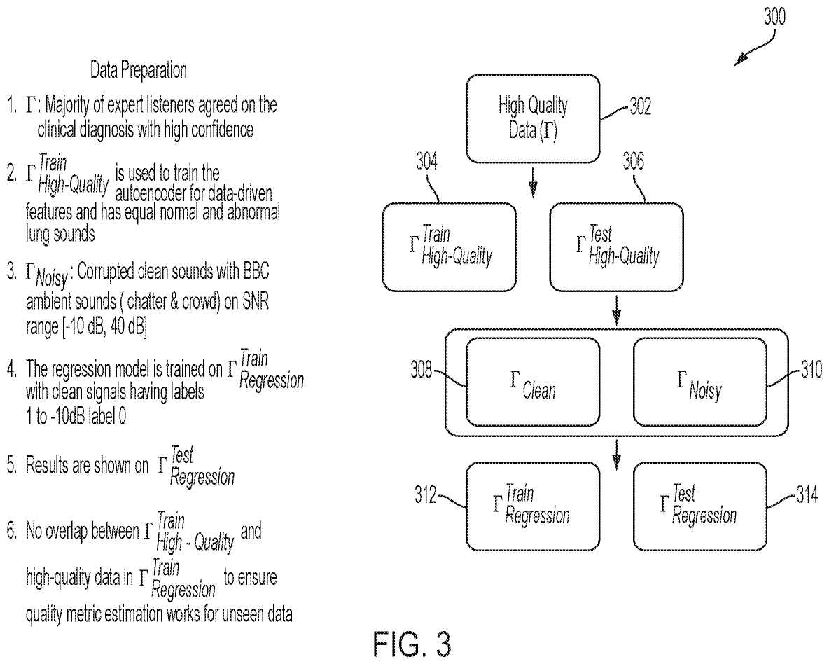

FIG. 3 shows a method 300 for data preparation. At 302, high quality data (Γ) is obtained, where Γ represents that a majority of expert listeners agreed on the clinical diagnosis with high confidence. At 304 and 306, the high quality data (Γ) is divided into Γ_{High-quality}^{Train} that is used to train the autoencoder for data-driven features and has equal normal and abnormal lung sounds and Γ_{High-Quality}^{Test}. At 308 and 310, Γ_{Clean} and Γ_{Noisy} are respectively obtained from Γ_{High-Quality}^{Test} where Γ_{Noisy} is corrupted clean sounds with BBC ambient sounds (chatter & crowd) on SNR range [−10 dB, 36 dB]. At 312 and 314, Γ_{Regression}^{Train} and Γ_{Regression}^{Test} are obtained from Γ_{Clean} and Γ_{Noisy}. The regression model is trained on Γ_{Regression}^{Train} with clean signals having labels 1 to −5 dB label 0. The results are shown on Γ_{Regression}^{Test}. This is no overlap between Γ_{High-Quality}^{Train} and high-quality data in Γ_{Regression}^{Train} to ensure quality metric estimation works for unseen data

A convolutional neural network (CNN) can be used as an autoencoder, and trained on auditory spectrograms generated from two second audio segments from the training dataset. The CNN can be a 3-, 4-, or 5-layer autoencoder. Other types of neural networks or machine/computer learning algorithms can also be used. The network learns filters that get activated if driven by certain auditory cues, thereby producing 2-dimensional activation map. In a 3-layer autoencoder example, the first two layers act as an encoder with the first layer extracting patches and second layer performing a non-linear mapping onto a low dimensional feature space; the third layer decodes the features back to the original spectrogram.

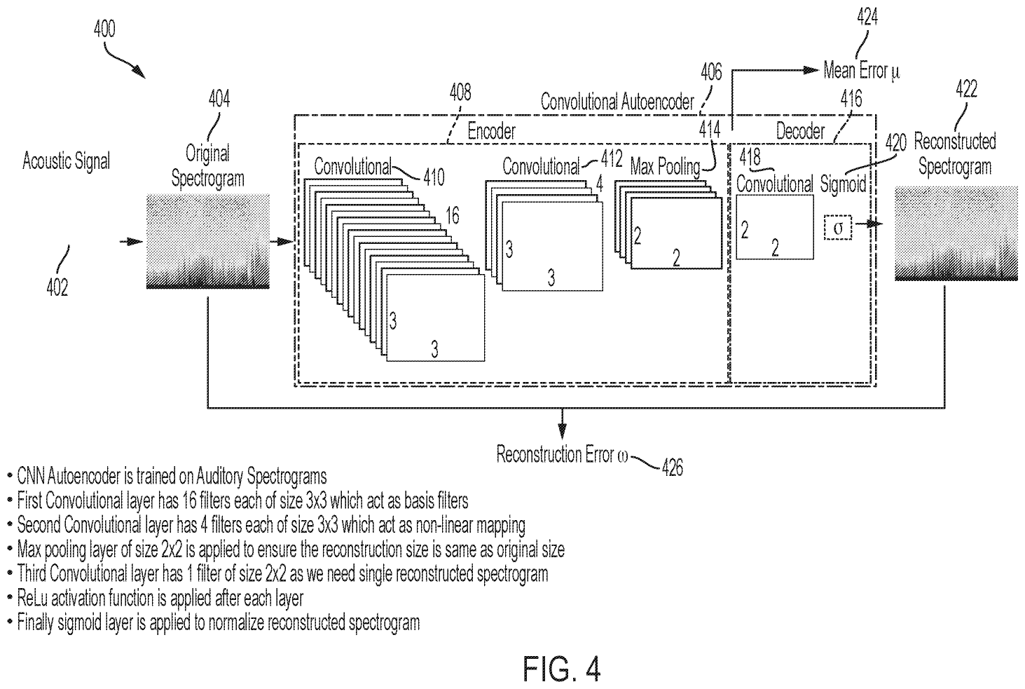

FIG. 4 shows the processing of the acoustic signal 400 using the three-layer convolutional autoencoder 406. The CNN Autoencoder is trained on auditory spectrograms. At 402, an acoustic signal is provided, which is then converted to an original spectrogram at 404. The original spectrogram is then provided to the convolutional autoencoder 406. The convolution autoencoder 406 comprises an encoder 408, which comprises a first convolutional layer 410, a second convolutional layer 412, and a pooling layer 414. The first convolutional layer 410 has 16 filters each of size 3×3 which act as basis filters. The second convolutional layer has 4 filters each of size 3×3 which act as non-linear mapping. The maximum pooling layer of size 2×2 is applied to ensure the reconstruction size is same as original size. A ReLu activation function is applied after each layer. A decoder 416 consists of a third convolutional layer 418 and an activation function 420. The third convolutional layer has 1 filter of size 2×2 as a single reconstructed spectrogram is desired. A sigmoid layer 420 is applied to normalize reconstructed spectrogram 422.

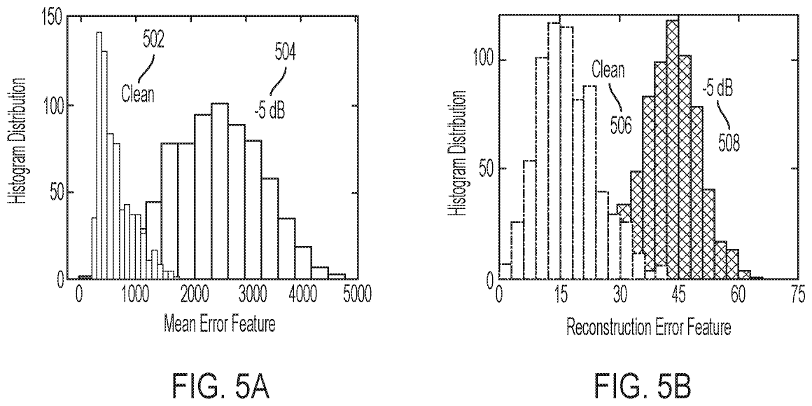

Once the convolution autoencoder 406 trained, two parameters are extracted from this network, and used to supplement the signal-centered features in our measure of lung quality. The first parameter is mean feature error (μ) 424. After passing a spectrogram (32×128 dimensions) through the encoder (first two layers of the CNN), a dense low dimensional (32×32) embedding is obtained. An average of all the training CNN embeddings acted as a high-quality data low-dimensional ‘template’. The L2 distance of the unsupervised features of the test data Γ_{Regression}^{Test} from the average feature template is taken as their corresponding mean feature error. FIGS. 5A and 5B show embedded features across different SNR values. FIG. 5A shows the distribution of this mean error (μ) for high-quality signals at 502. Overlaid on the same histogram is the distribution of mean errors obtained from −5 dB at 504. The second parameter is reconstruction error (w) 426. Assuming a good quality lung sound would be more similar to high-quality data and gives better reconstruction with the autoencoder trained on clean data, the L2 distance of the reconstructed spectrogram with the original spectrogram is considered as the second embedding feature. The reconstruction errors of −5 dB SNR sounds at 508 exhibit a clear rightward shift from clean signals at 506 in FIG. 5B.

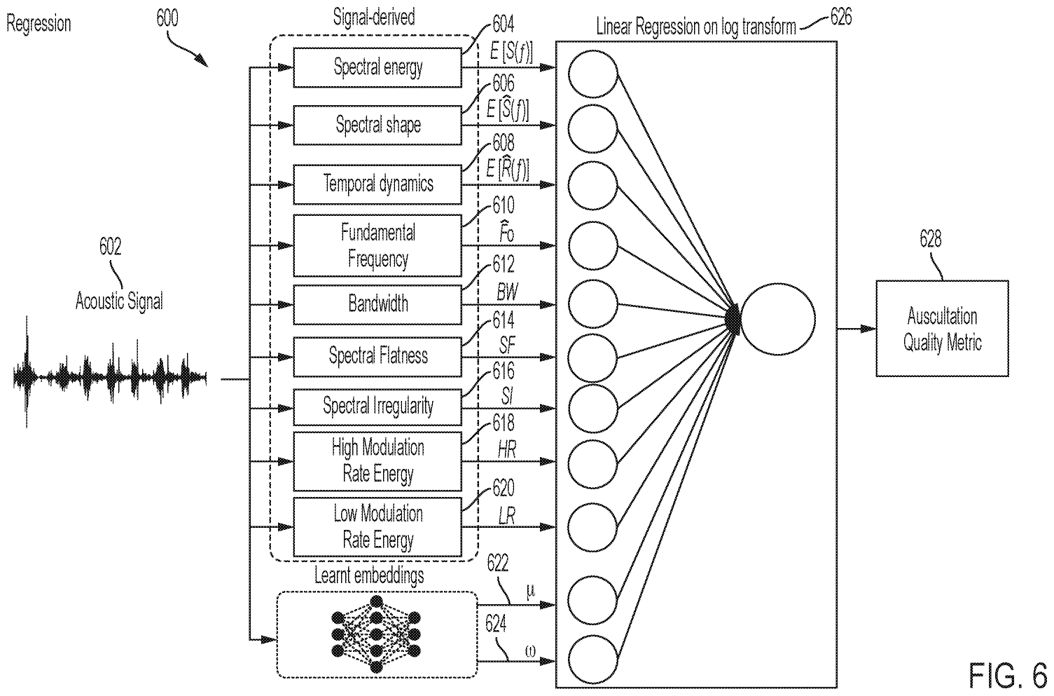

Both signal-centric and learnt features (using the autoencoder) are combined together to yield an overall quality metric. FIG. 6 shows a regression block diagram 600 for determining the overall quality metric. The eleven features were integrated using a multivariate linear regression performed on the log transformation of the features. The regression labels for Γ_{Regression}^{Train} ranged from 0 to 1 with 0 assigned to the −5 dB signal-to noise ratio values and 1 to the un-corrupted lung sounds. 10 dB and 20 dB SNR audio clippings were given intermediate labels.

As shown in FIG. 6, eleven signals are extracted from an acoustic signal 602. The eleven signals comprise a spectral energy E[S(f)] 604, a spectral shape E[Ŝ(f)] 606, a temporal dynamics E[\hat{R}(f)] 608, a fundamental frequency \hat{F}₀ 610, a bandwidth BW 612, a spectral flatness SF 614, a spectral irregularity SI 616, a high modulation rate energy HR 618, a low modulation rate energy LR 620, and two from learnt embeddings μ 622 and ω 624, as discussed above from the convolution autoencoder 406. The six signals are processed by a linear regression on log transform 626 to yield the auscultation quality metric 628.

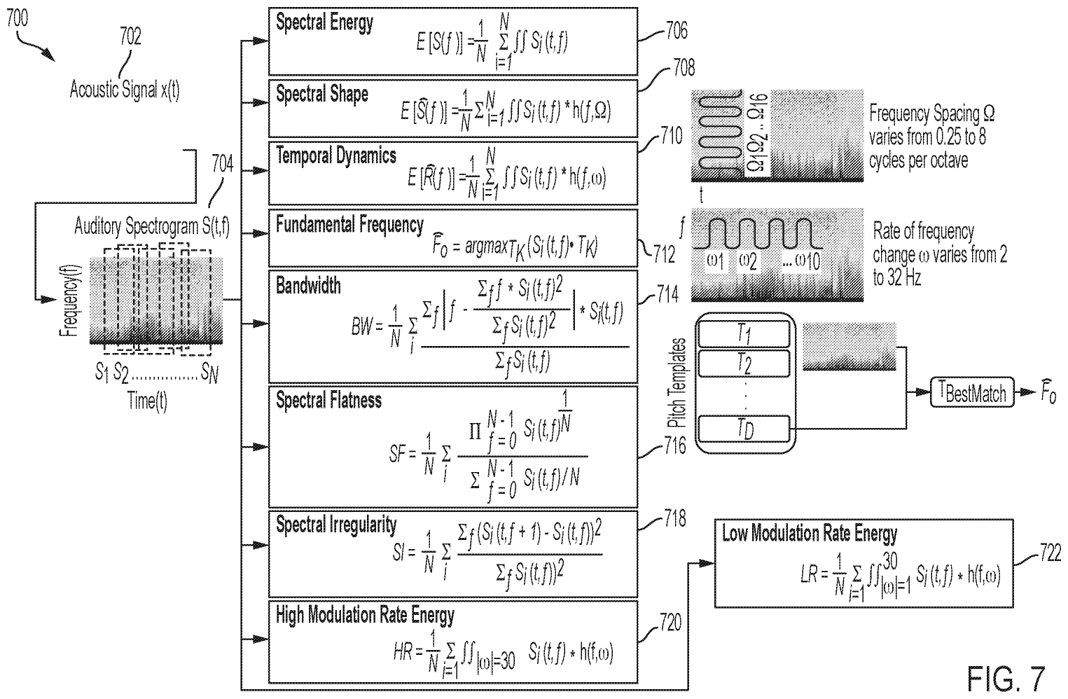

FIG. 7 shows a block diagram 700 of the regression parameters of FIG. 6. An auditory spectrogram S(t,f) 704 is obtained from an acoustic signal x(t) 702. Nine signals comprising spectral energy 706, spectral shape 708, temporal dynamics 710, fundamental frequency 712, bandwidth 714, spectral flatness 716, spectral irregularity 718, high modulation rate energy 720, and low modulation rate energy 722 are extracted from the auditory spectrogram S(t,f) 704.

The spectral energy 706 is represented by

$$E [ S ( f ) ] = \frac{1}{N} {\sum }_{i = 1}^{N} \int \int {S}_{i} ( t , f )$$

The spectral shape 708 is represented by:

$$E [ \hat{S} ( f ) ] = \frac{1}{N} {\sum }_{i = 1}^{N} \int \int {S}_{i} ( t , f ) * h ( t , Ω )$$

where the frequency spacing Ω varies from 0.25 to 8 cycles per octave.

The temporal dynamics 710 is represented by:

$$E [ \hat{R} ( f ) ] = \frac{1}{N} {\sum }_{i = 1}^{N} \int \int {S}_{i} ( t , f ) * h ( f , ω )$$

where the rate of frequency change w varies from 2 to 32 Hz.

The fundamental frequency 712 is represented by:

\hat{F}₀=(argmax_{T}_{ₖ}(Sᵢ(t,f)·Tₖ)

where the pitch templates T₁, T₂, . . . , Tₖ. the pitch templates T₁, T₂, . . . Tₖ are harmonic templates that evaluate the best match with Si(t,f) and yield the fundamental frequency \hat{F}₀.

The bandwidth 714 is represented by:

$$B W = \frac{1}{N} {\sum }_{i} \frac{{\sum }_{f} \vert f - \frac{{\sum }_{f} f * {{S}_{i} ( t , f )}^{2}}{{\sum }_{f} {{S}_{i} ( t , f )}^{2}} \vert * {S}_{i} ( t , f )}{{\sum }_{f} {S}_{i} ( t , f )}$$

The spectral flatness 716 is represented by:

$$S F = \frac{1}{N} {\sum }_{i} \frac{{\prod }_{f = 0}^{N - 1} {{S}_{i} ( t , f )}^{\frac{1}{N}}}{{\sum }_{f = 0}^{N - 1} \frac{{S}_{i} ( t , f )}{N}}$$

The spectral irregularity 718 is represented by:

$$SI ( t ) = \frac{1}{N} {\sum }_{i} \frac{{\sum }_{f} {( {S}_{i} ( t , f + 1 ) - {S}_{i} ( t , f ) )}^{2}}{{\sum }_{f} {{S}_{i} ( t , f )}^{2}}$$

The high modulation rate energy 720 is represented by:

$$H R = \frac{1}{N} {\sum }_{i = 1} \int {\int }_{\vert ω \vert = 30}^{128} {S}_{i} ( t , f ) * h ( f , ω )$$

The low modulation rate energy 722 is represented by:

$$L R = \frac{1}{N} {\sum }_{i = 1} \int {\int }_{\vert ω \vert = 1}^{30} {S}_{i} ( t , f ) * h ( f , ω )$$

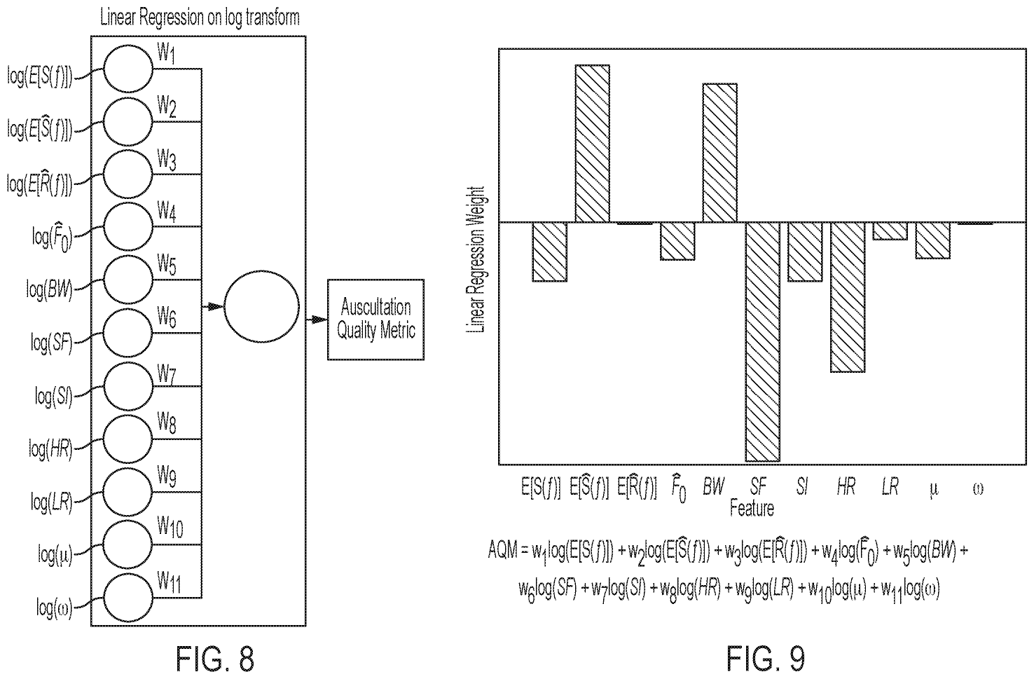

FIG. 8 shows the linear regression on log transform and the auscultation quality metric of FIG. 6 in more detail. The auscultation quality metric (AQM) can be given by:

AQM=w₁ log(E[S(f)])+w₂ log(E[Ŝ(f)])+w₃ log(E[\hat{R}(f)])+w₄ log(\hat{F}₀)+w₅ log(BW)+w₆ log(SF)+w₇ log(SI)+w₈ log(HR)+w₉ log(LR)+w₁₀ log(μ)+w₁₁ log(ω)

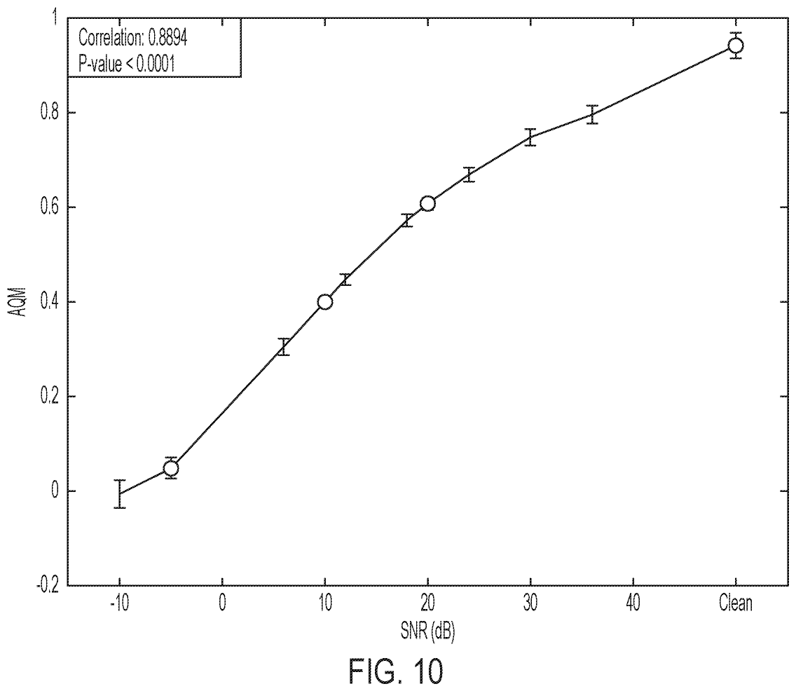

FIG. 9 shows a plot of linear regression weight versus features. The obtained quality metric shows a strong correlation of 0.89±0.0039 on a 10-fold cross validation across the span of signal to noise ratios with a high very high significance (p-value <0.0001). The compliance of this correlation by lung sounds in Γ_{Regression}^{Test} with additional signal to noise ratios which were not included in Γ_{Regression}^{Train} further validates the quality metric as shown in FIG. 10. FIG. 10 shows average Auscultation Quality Metric (AQM) from 0 to 1 vs signal to noise ratio (SNR) in dB with the circles indicating the SNR values included in the Γ_{Regression}^{Train}. The error bars represent variance of AQM for each SNR.

Often times, access to the recorded lung sound is only available and not the surrounding ambient noise. This makes the estimation of noise content in the signal rather difficult. Since the lung sounds contain adventitious patterns which have similar spectral and temporal patterns as the ambient noise, it is difficult to gauge the quality by the signal alone. In this work, by creating a template of what a high-quality lung signal sounds like irrespective of whether they are normal or abnormal (wheezes and crackles), a quality metric can be estimated.

Auditory salience features can be used which account for the noise content as well unsupervised embedded features based on the clean template which justify the presence of the adventitious sound patterns. Further analysis can be done on testing the potential use of this metric as a preprocessing criteria for automated lung sound analyses. Also, if integrated with digital devices, data curation could be made more efficient by alerting the physician of the bad quality immediately to record again.

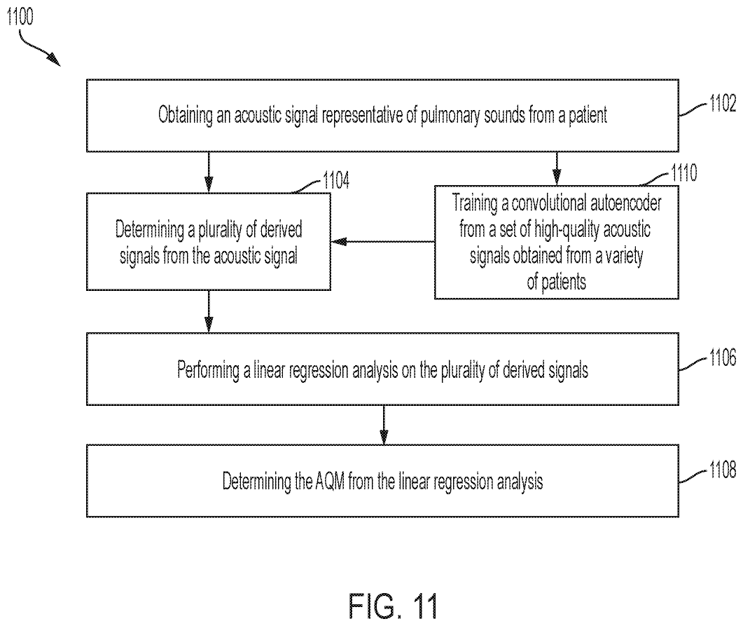

FIG. 11 show a computer-implemented method for determining an auscultation quality metric (AQM) 1100, according to examples of the present disclosure. The computer-implemented method 1100 comprises obtaining an acoustic signal representative of pulmonary sounds from a patient, as in 1102. The computer-implemented method 1100 continues by determining a plurality of derived signals from the acoustic signal, as in 1104. The plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, a low modulation rate energy signal, or various combinations thereof. The mean error signal and the reconstruction error signal are obtained from a trained neural network. In some examples, the trained neural network can be a trained convolutional autoencoder. The trained convolutional autoencoder can comprise three layers or other configurations autoencoders, such as a four-layer autoencoder or a five-layer autoencoder. The computer-implemented method 1100 continues by performing a regression analysis, such as a linear regression analysis, on the plurality of derived signals, as in 1106. The computer-implemented method 1100 continues by determining the AQM from the regression analysis, as in 1108. The AQM ranges from 0 to 1 where 0 represents the lowest quality and 1 represents the highest quality for the acoustic signal that is obtained. In some examples, the computer-implemented method 1100 can further comprise training a convolutional autoencoder from a set of high-quality acoustic signals obtained from a variety of patients.

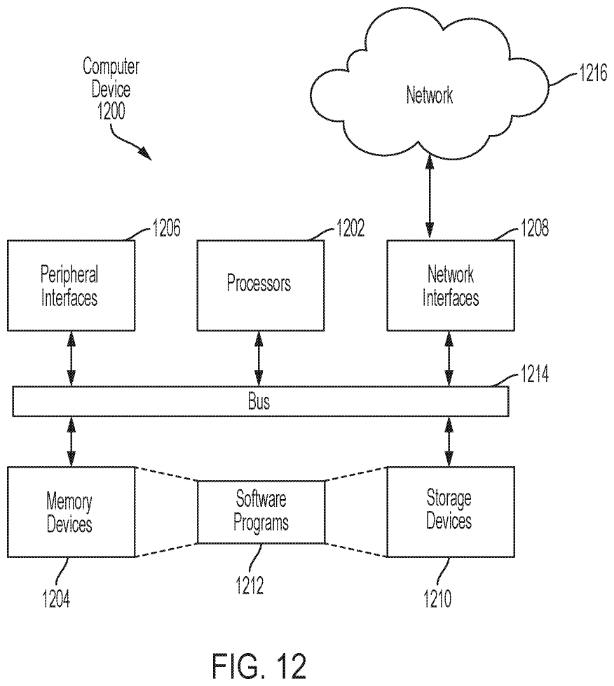

FIG. 12 is an example of a hardware configuration for a computer device 1200, which can be used to perform one or more of the processes described above. The computer device 1200 can be any type of computer devices, such as desktops, laptops, servers, etc., or mobile devices, such as smart telephones, tablet computers, cellular telephones, personal digital assistants, etc. As illustrated in FIG. 12, the computer device 1200 can include one or more processors 1202 of varying core configurations and clock frequencies. The computer device 1200 can also include one or more memory devices 1204 that serve as a main memory during the operation of the computer device 1200. For example, during operation, a copy of the software that supports the above-described operations can be stored in the one or more memory devices 1204. The computer device 1200 can also include one or more peripheral interfaces 1206, such as keyboards, mice, touchpads, computer screens, touchscreens, etc., for enabling human interaction with and manipulation of the computer device 1200.

The computer device 1200 can also include one or more network interfaces 1308 for communicating via one or more networks, such as Ethernet adapters, wireless transceivers, or serial network components, for communicating over wired or wireless media using protocols. The computer device 1200 can also include one or more storage devices 1210 of varying physical dimensions and storage capacities, such as flash drives, hard drives, random access memory, etc., for storing data, such as images, files, and program instructions for execution by the one or more processors 1202.

Additionally, the computer device 1200 can include one or more software programs 1212 that enable the functionality described above. The one or more software programs 1212 can include instructions that cause the one or more processors 1202 to perform the processes, functions, and operations described herein, for example, with respect to the process of described above. Copies of the one or more software programs 1212 can be stored in the one or more memory devices 1204 and/or on in the one or more storage devices 1210. Likewise, the data utilized by one or more software programs 1212 can be stored in the one or more memory devices 1204 and/or on in the one or more storage devices 1210. Peripheral interface 1206, one or more processors 1202, network interfaces 1208, one or more memory devices 1204, one or more software programs, and one or more storage devices 1210 communicate over bus 1214.

In implementations, the computer device 1200 can communicate with other devices via a network 1216. The other devices can be any types of devices as described above. The network 1216 can be any type of network, such as a local area network, a wide-area network, a virtual private network, the Internet, an intranet, an extranet, a public switched telephone network, an infrared network, a wireless network, and any combination thereof. The network 1216 can support communications using any of a variety of commercially-available protocols, such as TCP/IP, UDP, OSI, FTP, UPnP, NFS, CIFS, AppleTalk, and the like. The network 1216 can be, for example, a local area network, a wide-area network, a virtual private network, the Internet, an intranet, an extranet, a public switched telephone network, an infrared network, a wireless network, and any combination thereof.

The computer device 1200 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of the computer device 1200 as described above need not be enclosed within a single enclosure or even located in close proximity to one another. Those skilled in the art will appreciate that the above-described componentry are examples only, as the computer device 1200 can include any type of hardware componentry, including any necessary accompanying firmware or software, for performing the disclosed implementations. The computer device 1200 can also be implemented in part or in whole by electronic circuit components or processors, such as application-specific integrated circuits (ASICs) or field-programmable gate arrays (FPGAs).

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with the embodiments disclosed herein can be implemented or performed with a general purpose processor, a digital signal processor (DSP), an application specific integrated circuit (ASIC), a field programmable gate array (FPGA), cryptographic co-processor, or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

What is claimed is:

1. A computer-implemented method for determining an auscultation quality metric (AQM), the computer-implemented method comprising:
obtaining an acoustic signal representative of pulmonary sounds from a patient;
determining a plurality of derived signals from the acoustic signal;
performing a regression analysis on the plurality of derived signals; and
determining the AQM from the regression analysis.

2. The computer-implemented method of claim 1, wherein the plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, or a low modulation rate energy signal.

3. The computer-implemented method of claim 2, wherein the mean error signal and the reconstruction error signal are obtained from a trained neural network.

4. The computer-implemented method of claim 3, wherein the trained neural network is a trained convolutional autoencoder.

5. The computer-implemented method of claim 4, wherein the trained convolutional autoencoder is a three-layer autoencoder, a four-layer autoencoder, or a five-layer autoencoder.

6. The computer-implemented method of claim 1, further comprising training a convolutional autoencoder from a set of high-quality acoustic signals obtained from a variety of patients.

7. The computer-implemented method of claim 1, wherein the AQM ranges from 0 to 1 where 0 represents the lowest quality and 1 represents the highest quality for the acoustic signal that is obtained.

8. A computer system comprising:
a hardware processor;
a non-transitory computer readable medium comprising instructions that when executed by the hardware processor perform a method for determining an auscultation quality metric (AQM), comprising:
obtaining an acoustic signal representative of pulmonary sounds from a patient;
determining a plurality of derived signals from the acoustic signal;
performing a regression analysis on the plurality of derived signals; and
determining the AQM from the regression analysis.

9. The computer system of claim 8, wherein the plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, or a low modulation rate energy signal.

10. The computer system of claim 9, wherein the mean error signal and the reconstruction error signal are obtained from a trained neural network.

11. The computer system of claim 10, wherein the trained neural network is a trained convolutional autoencoder.

12. The computer system of claim 11, wherein the trained convolutional autoencoder is a three-layer autoencoder, a four-layer autoencoder, or a five-layer autoencoder.

13. The computer system of claim 8, wherein the hardware processor is further configured to execute the method comprising training a convolutional autoencoder from a set of acoustic signal obtained from a variety of patients.

14. The computer system of claim 8, wherein the AQM ranges from 0 to 1 where 0 represents the lowest quality and 1 represents the highest quality for the acoustic signal that is obtained.

15. A non-transitory computer readable medium comprising instructions that when executed by a hardware processor perform a method for determining an auscultation quality metric (AQM), method comprising:
obtaining an acoustic signal representative of pulmonary sounds from a patient;
determining a plurality of derived signals from the acoustic signal;
performing a regression analysis on the plurality of derived signals; and
determining the AQM from the regression analysis.

16. The non-transitory computer readable medium of claim 15, wherein the plurality of derived signals comprise a spectral energy signal, a spectral shape signal, a temporal dynamics signal, a fundamental frequency signal, a mean error signal, a reconstruction error signal, a bandwidth signal, a spectral flatness signal, a spectral irregularity signal, a high modulation rate energy signal, or a low modulation rate energy signal.

17. The non-transitory computer readable medium of claim 16, wherein the mean error signal and the reconstruction error signal are obtained from a trained neural network.

18. The non-transitory computer readable medium of claim 17, wherein the trained neural network is a trained convolutional autoencoder.

19. The non-transitory computer readable medium of claim 18, wherein the trained convolutional autoencoder is a three-layer autoencoder, a four-layer autoencoder, or a five-layer autoencoder.

20. The non-transitory computer readable medium of claim 15, wherein the method further comprises training a convolutional autoencoder from a set of acoustic signal obtained from a variety of patients.

21. The non-transitory computer readable medium of claim 15, wherein the AQM ranges from 0 to 1 where 0 represents the lowest quality and 1 represents the highest quality for the acoustic signal that is obtained.
